(19) United States
(12) Patent Application Publication
(10) Pub. No.: US 2023/0057865 A1
(43) Pub. Date: Feb. 23, 2023

(54) ULTRAVIOLET LIGHT FIXTURE

(71) Applicants: Thomas W. Gow, Lake Preston, SD (US); Matthew James Gow, Coon Rapids, MN (US); Thomas Jason Gow, Andover, MN (US)

(72) Inventors: Thomas W. Gow, Lake Preston, SD (US); Matthew James Gow, Coon Rapids, MN (US); Thomas Jason Gow, Andover, MN (US)

(21) Appl. No.: 17/889,176

(22) Filed: Aug. 16, 2022

Related U.S. Application Data
(60) Provisional application No. 63/234,075, filed on Aug. 17, 2021.

(51) Int. Cl. A61L 2/10

(52) U.S. Cl. CPC A61L 2/10; A61L 2202/11; A61L 2202/25; A61L 2202/14

(57) ABSTRACT

An ultraviolet (UV) light fixture apparatus, according to an example, includes a fixture head apparatus including a front panel, a rotatable fixture head unit, and at least one UV lamp, wherein the front panel is to be mounted substantially flush with a mounting surface with the rotatable fixture head unit positioned behind the front panel within a recess behind the mounting surface. The UV light fixture apparatus includes a motor to cause rotation of the rotatable fixture head unit to switch the rotatable fixture head unit between a concealed state in which the at least one UV lamp is not visible and an exposed state in which the at least one UV lamp is visible through an opening in the front panel.

### CROSS-REFERENCE TO RELATED APPLICATIONS

This U.S. Patent application claims the benefit of the filing date of U.S. Provisional Patent Application Ser. No. 63/234,075, filed Aug. 17, 2021, the entire teachings of which are incorporated herein by reference.

### BACKGROUND

An ultraviolet (UV) lighting system may be used to disinfect an area, such as a room in a building. However, the operation of the UV type of disinfection lighting may be damaging to humans if they are exposed to the light which includes the invisible lighting frequency spectrum. Some UV disinfection lighting fixtures may be obtrusive and interrupt the aesthetic beauty of interior designs.

For these and other reasons, a need exists for the present invention.

### SUMMARY

An example is directed to an ultraviolet (UV) light fixture apparatus, which includes a fixture head apparatus including a front panel, a rotatable fixture head unit, and at least one UV lamp, wherein the front panel is to be mounted substantially flush with a mounting surface with the rotatable fixture head unit positioned behind the front panel within a recess behind the mounting surface. The UV light fixture apparatus includes a motor to cause rotation of the rotatable fixture head unit to switch the rotatable fixture head unit between a concealed state in which the at least one UV lamp is not visible and an exposed state in which the at least one UV lamp is visible through an opening in the front panel.

### BRIEF DESCRIPTION OF THE DRAWINGS

The accompanying drawings are included to provide a further understanding of embodiments and are incorporated in and constitute a part of this specification.

The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 is a diagram illustrating a perspective view of an ultraviolet (UV) light fixture apparatus prior to installation in a mounting surface according to an example.

FIG. 2 is a diagram illustrating a perspective view of a UV light fixture apparatus prior to installation in a mounting surface according to another example.

FIG. 3 is a diagram illustrating a perspective view of the back box of a UV light fixture apparatus mounted to a stud of a mounting surface according to an example.

FIG. 4 is a diagram illustrating a side view of a UV light fixture apparatus prior to installation in a mounting surface according to an example.

FIG. 5 is a diagram illustrating a perspective view of the fixture head apparatus of a UV light fixture apparatus in a concealed position according to an example.

FIG. 6 is a diagram illustrating another perspective view of the fixture head apparatus shown in FIG. 5 in a concealed position according to an example.

FIG. 7 is a diagram illustrating another perspective view of the fixture head apparatus shown in FIG. 5 in an exposed position according to an example.

FIG. 8 is a diagram illustrating internal details of the fixture head apparatus shown in FIG. 5 according to an example.

FIG. 9 is an electrical schematic diagram illustrating electrical elements of the fixture head apparatus shown in FIG. 5 according to an example.

FIG. 10 is a diagram illustrating internal details of a fixture head apparatus according to another example.

FIG. 11 is a diagram illustrating a perspective view of the fixture head apparatus shown in FIG. 10 transitioning from a concealed position to an exposed position according to an example.

FIG. 12 is a diagram illustrating a perspective view of the fixture head apparatus shown in FIG. 10 in an exposed position according to an example.

FIG. 13 is a diagram illustrating a perspective view of a fixture head apparatus in an exposed position according to another example.

FIG. 14 is a block diagram illustrating elements of a UV light fixture apparatus according to an example.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

It is to be understood that the features of the various exemplary embodiments described herein may be combined with each other, unless specifically noted otherwise.

An example is directed to a concealed ultraviolet (UV) light fixture apparatus. The UV light fixture apparatus may be concealed and operated to automatically expose a lighting portion when needed for building room UV disinfection. The UV light fixture apparatus may be controlled based on a motion detection system.

Some examples disclosed herein are directed to a concealed UV light fixture apparatus with detector controls. In some examples, the light fixture is a concealed from view ceiling or wall mounted UV light fixture. The UV light fixture may be concealed and operated to be activated to expose the lighting portion when needed for building room UV disinfection modes that must operate daily.

The operation of the UV type of disinfection lighting itself may be damaging to humans if they are exposed to the light which includes the invisible lighting frequency spectrum. This disinfection lighting should only then operate when humans and other animals etc. are not present. The operation of the concealed UV light fixture door operation and activation of the UV light itself may be controlled by using an internal or external motion detection system using sonic or radio frequency (RF) or infrared detection system. The motion detection system may be integral within the light fixture unit and may allow motion from a concealed position using detectors that can include detector coverage that can pass through a non-ferrous concealed door assembly to maintain the aesthetics of the room. This detection system may over-ride the activation of the door rotation and UV light fixture activation. These detectors may also be located within the light fixture so that when the door is rotated, exposing the UV light fixture along with the detector, it will inhibit the UV light fixture from activation from operating if the detector senses a presence within the room. In addition, a photocell may also be used, either integral or remotely to over-ride the door operation and UV light fixture operation.

The concealed UV light fixture may also be connected to building management control systems to receive commands as to when to operate as part of an automatic scheduled programmable activation program. The management system can also receive the cycles of operation/duration/attempts of the UV light fixture and why and when the UV light fixture may have operated or not operated, and sense what detection may have not allowed the completed timed operation.

Other electrical apparatuses may be used within this concealed enclosure in addition to lighting purposes that would include motion detectors, and security alarm audio and visual annunciators. The concealed UV light fixture with detector(s) can operate as part of the building power branch circuit system or be self-contained with battery back-up for emergency power outages.

Some examples of the UV light fixture system may include a gear drive mechanism that rotates the concealed door UV light fixture and detector(s) from the concealed door position to the exposed disinfection position and return to the closed concealed position in both directions by using a reversing relay circuit to reverse the polarity of the DC gear drive motor assembly. Limit switches may make contact de-energizing the gear drive mechanism when the door has successfully reached the respective closed concealed door position or the exposed UV light fixture and detector(s) positions.

The door and door frame assembly features may be flush concealed mounted within common building wall and ceiling construction materials including gypsum board walls and ceilings, suspended ceilings, brick, concrete, tile, and marble. Some examples disclosed herein are directed to a virtually invisible concealed UV disinfecting lighting system enclosure that preserves the integrity of architectural or interior designs, without sacrificing security and performance.

Some UV disinfection lighting fixtures may be obtrusive and interrupt the aesthetic beauty of interior designs. Some examples disclosed herein provide a beautiful and vandal-resistant alternative to visible surface mount disinfection lighting. Only when the commercial or residential UV disinfecting lighting is activated, does the UV disinfection light become visible, rotating 180 degrees to begin operation. The result is a clean, almost invisible installation that does not conflict with interiors.

The UV lighting system enclosure can conceal virtually all types and brands of UV disinfection lighting fixtures, and may be used for commercial and residential applications. Some examples may have a white powder coat finish or may be customized in the field using paint, wallpaper, or applique finishes. The enclosures may be compact, completely self-contained units that are vandal resistant. The enclosure may be used for wall or ceiling installations, including gypsum board, plaster, concrete, brick, or acoustical tile. The enclosure includes fully automatic operation where the door is operated by a 12 volt DC motor, with an integrated gear head operated by the building's 120 VAC, 60 Hz branch circuiting. The UV lighting system may be manually controlled or integrated into the building management control networks via CAT5 cable system.

The UV light fixture apparatus may utilize a recessed flange for flush mounting in either a wall or ceiling. 12 VDC power may be derived from security panel through conduit knockouts on the recessed enclosure. The door, UV disinfection lamp, and motor drive assembly may be inserted into the recessed enclosure. The assembly may then be secured in place with two concealed bolts that allow for adjustment to the wall or ceiling thickness. The units may be implemented for gypsum board, suspended ceiling, and surface mount.

For new projects, a single enclosure may be mounted before the wall or ceiling material is installed. For retrofit/remodeling projects, a multi-section enclosure may be used with a power input of 120 VAC, 60 Hz, 3.6 amps.

The unit may be stored in a retracted position during non-activated conditions. Upon activating the UV lamp lighting system, the unit may be deployed into the open position and the UV lamp system energized. Deactivation of the UV lighting system may cause the unit to retract to the stored position and de-energize the UV lighting system. The motor may be a 12 volt sealed permanent magnet type. The electronics for the unit may be a solid-state design.

Control options for the UV lighting system include controlling through BACnet, including switching the unit on/off through the facility BACnet, and programming through the Building Automation Software system dashboard. The unit may be manually switched on/off for a single default or commissioned cycle through a switched or a remote-controlled power input. Commissioning may be done at the unit using an Ethernet port and a simple setup wizard.

In some examples, the UV lighting system includes safety and occupancy sensors. Redundant safety systems ensure that the unit will not activate while the room is occupied and will immediately suspend operations if motion is detected in a previously unoccupied room. If interrupted, the unit will resume its programmed cleaning cycle once motion is no longer detected.

The unit may be provided to operate in either a BACnet integrated mode or a manually commissioned mode. In some examples, the modes may be configured at the factory and cannot be changed on the device. In the BACnet integration mode, disinfection-on-demand operation may be programmed through the Building Automation Software (BAS) through BACnet. The cycle time, cycle repeat, cycle schedule and motion sensor settings and unit on/off and initial activation may be controlled through the system dashboard. In some examples, devices in the BACnet integration mode are initially activated through the BAS software in order to operate. By default in some examples, this mode may have safety features deactivated. In the manually commissioned mode, the unit may be manually commissioned with commissioning for disinfection-on-demand including cycle time, cycle repeat and cycle schedule settings.

The UV lighting system disinfects a whole room, delivering disinfection from both wall and ceiling locations above. It emits broad spectrum UV light to eliminate up to 99.9% of the bacteria, viruses, and fungi in a space. In rooms that have been cleaned of mold, the unit will help control return growth if used routinely. The UV lighting system may use broad spectrum UV-C(200-280 nm) germicidal, UV-B (280-320 nm) germicidal, and UV-A (320-400 nm) antibacterial light to kill bacteria and viruses. The system may be programmed manually or integrated into a building automation system through BACnet. Some examples use a single UV light engine with pulsed Xenon lamp for ten feet by ten feet (i.e., 100 square feet) coverage area. The system may use a flash interval of one UV flash every six seconds. The system may use an input voltage of 110 VAC, with a max current of 3.5 amps, a total power of 25 W per standard 30-minute cycle, and a power connection of two wires and ground. In some examples, the UV lighting system weighs six pounds, and has a brushed aluminum housing finish. The optics of the system may include a single UV light engine with transmissive UV lens and pulsed Xenon UV lamp, and a beam angle of 170 degrees.

In some examples, the unit includes a back box enclosure for 120 VAC, 60 Hz input terminations. A non-GF110V outlet may be switched out for convenience. The unit may also be powered by a Bluetooth controlled smart unit, eliminating the need to install a switch and using instead a smartphone based application to turn the unit on/off through the smart outlet. Cycles for the unit may include a standard cycle (e.g., 30 minutes/day), a deep cycle (e.g., 1 hour/day), and a terminal cycle (e.g., 2 hours/day).

The concealed design of the UV light fixture system improves building room aesthetics, and the design protects components from vandalism. Hermetically sealed door and door frame design of the UV light fixture system secures the hygienics of the interior enclosure during inactive mode of operation. The sealed door assembly provides damp and wet location area installations. The concealed sealed design maintains internal concealed components for reducing cleaning maintenance and reducing time and labor costs of repeated cleaning as with exposed versions.

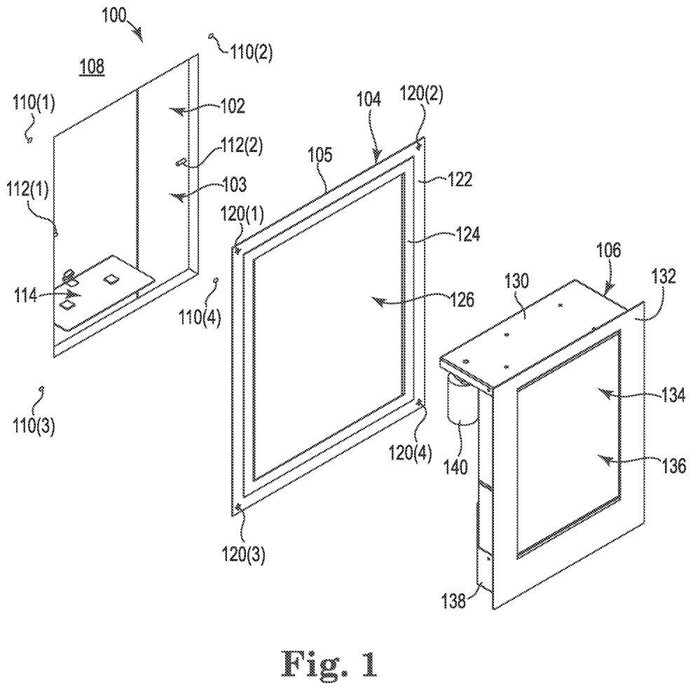

FIG. 1 is a diagram illustrating a perspective view of a UV light fixture apparatus 100 prior to installation in a mounting surface 108 according to an example. In the illustrated example, the mounting surface 108 is a wall. Apparatus 100 includes a back box 102, a flush mounting system 104, and a fixture head apparatus 106.

Common building construction uses wallboard (also referred to as drywall or gypsum board) or other types of panels mounted on studs for the walls, or on joists for ceilings to define individual occupancy spaces (rooms, halls, etc.). The spaces between the studs or joists behind the panels are usually void or occupied by insulation. In commercial buildings and multiple dwellings, some ceilings use panels that fit into metal frames suspended from I-beams or poured concrete layers. For the sake of simplicity, all of these various types of room-defining panels having voids behind them will hereafter be referred to as “walls”.

In some examples, apparatus 100 is configured to be installed in wall 108. Wall 108 may be supported by studs, and may include a rectangular-shaped opening or recess 103, which is sized to receive a rear portion of the fixture head apparatus 106 therein. Back box 102 is positioned behind the wall 108 and includes a front panel with an opening that faces the opening 103 in the wall 108. The opening in the front panel of the back box 102 allows the rear portion of the fixture head apparatus 106 to be received into the interior of the back box 102. The back box 102 includes mounting posts 112(1)-112(2) (collectively referred to as mounting posts 112) to facilitate mounting the fixture head apparatus 106 to the back box 102. Mounting post 112(1) extends from an inner surface of a left panel of the back box 102 toward an interior of the box 102. Mounting post 112(2) extends from an inner surface of a right panel of the back box 102 toward an interior of the box 102. The back box 102 also includes a cradle assembly 114 attached to an interior surface of a back panel of the back box 102.

Fixture head apparatus 106 includes a top panel 130, a left side panel 138, a front panel 132, a rotatable fixture head unit 136, and a motor 140. The front panel 132 includes a rectangular-shaped opening 134 inside of which is positioned the rotatable fixture head unit 136. The motor 140 is attached to a bottom surface of the top panel 130.

Flush mounting system 104 includes a mounting plate 105. Mounting plate 105 includes a rectangular-shaped opening 126, an inner region 124 directly adjacent to and surrounding the opening 126, and an outer region 122 directly adjacent to and surrounding the inner region 124. The inner region 124 is slightly recessed with respect to the outer region 122. The rear portion of the fixture head apparatus 106 has a smaller size than the opening 126 formed in the mounting plate 105, which allows the rear portion of the fixture head apparatus 106 to be inserted through the opening 126 and be positioned on a back side of the mounting plate 105. The front panel 132 of the fixture head apparatus 106 is larger than the opening 126, but smaller than the mounting plate 105. The portion of the back surface of the front panel 132 adjacent the perimeter of the front panel 132 rests against the inner region 124 of the mounting plate 105 when the rear portion of the fixture head apparatus 106 has been inserted completely through the opening 126.

The back side of the outer region 122 of the mounting plate 105 may be attached to a front side of the wall 108. When attached to the wall 108, the inner region 124 of the mounting plate 105 partially covers the outer edges of the opening 103. Wall 100 includes four holes 110(1)-110(4) (collectively referred to as holes 110) formed therein around a perimeter of the opening 103. When mounting plate 105 is being attached to the wall 108, holes 120(1)-120(4) (collectively referred to has holes 120) formed near the four corners of the mounting plate 105 respectively align with holes 110(1)-110(4) formed in the wall 108. Attachment structures (e.g., pins, screws, bolts, or other attachment structures) may then be inserted through holes 120 and into holes 110 to attach the mounting plate 105 to the wall 108 and to help maintain the installed positioned of the fixture head apparatus 106.

After installation, the front surface of the front panel 132 is flush or nearly flush with the outer finished surface of the wall 108. After installation, the rear portion of the fixture head apparatus 106 (e.g., the portion behind the front panel 132) is positioned completely behind the outer surface of the wall 108. Apparatus 100 may be mounted into any of many wall materials. In addition to gypsum board, other wall materials where the apparatus 100 may be installed include brick and mortar, wood, concrete, fiberglass, and metal construction. Matching wall finishes may be applied to the wall, the mounting plate 105, and the front panel 132, including paint and wall paper fabric coverage, as well as a textured finished surface to match any other finished wall materials.

After mounting the apparatus 100 onto the wall 108, standard mudding of plaster compound and fiberglass taping may be applied overlapping the perimeter edges of the mounting plate 105 and the wall surface. This allows the apparatus 100 to be concealed on the same plane as the finished wall surface. The mounting plate 105 and the front panel 132 allow the application thereon of the same matching overall wall finished colors to blend these elements into each unique room finished wall or ceiling appearance. After installation, the apparatus 100 has an aesthetically pleasing appearance in regard to the desire of architects and interior designers because it is substantially concealed and blends in with the surrounding wall surface. The apparatus 100 has the ability to provide the best appearance regardless of the particular wall construction materials and textures, including gypsum board, wood, concrete block, brick, stone, marble, and others.

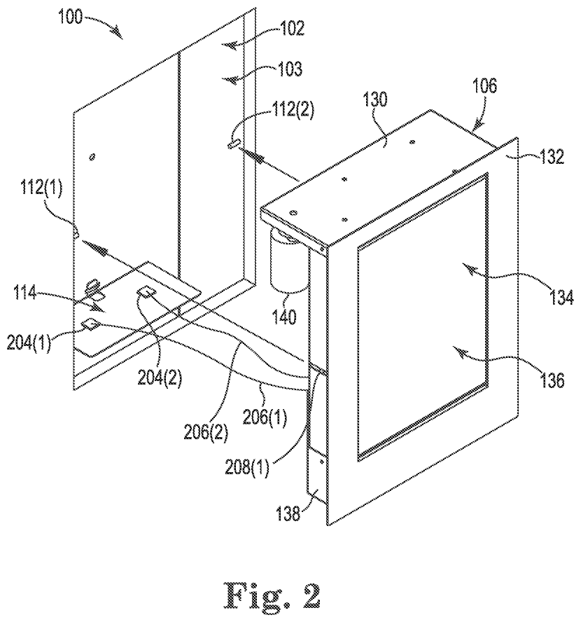

FIG. 2 is a diagram illustrating a perspective view of a UV light fixture apparatus 100 prior to installation in a mounting surface 108 according to another example. As shown in FIG. 2, the back box 102 includes a cradle assembly 114 attached to an interior surface of a back panel of the back box 102 via an attachment mechanism. The cradle assembly 114 includes electrical contacts 204(1)-204(2) (collectively referred to as electrical contacts 204). The electrical contacts 204 of the cradle assembly 114 may be electrically connected to electrical contacts of the fixture head apparatus 106 via wires 206(1)-206(2) (collectively referred to as wires 206). The opening in the front panel of the back box 102 allows the rear portion of the fixture head apparatus 106 to be received into the interior of the back box 102. As the fixture head apparatus 106 is inserted into the back box 102, the mounting post 112(1) of the back box 102 engages with an adjustable mounting slot 208(1) formed in the left side panel 138, and the mounting post 112(2) of the back box 102 engages with an adjustable mounting slot formed on the right side panel of the fixture head apparatus 106. The mounting posts 112 and the adjustable mounting slots help secure the rear portion of the fixture head apparatus 106 in proper position within the interior of the back box 102.

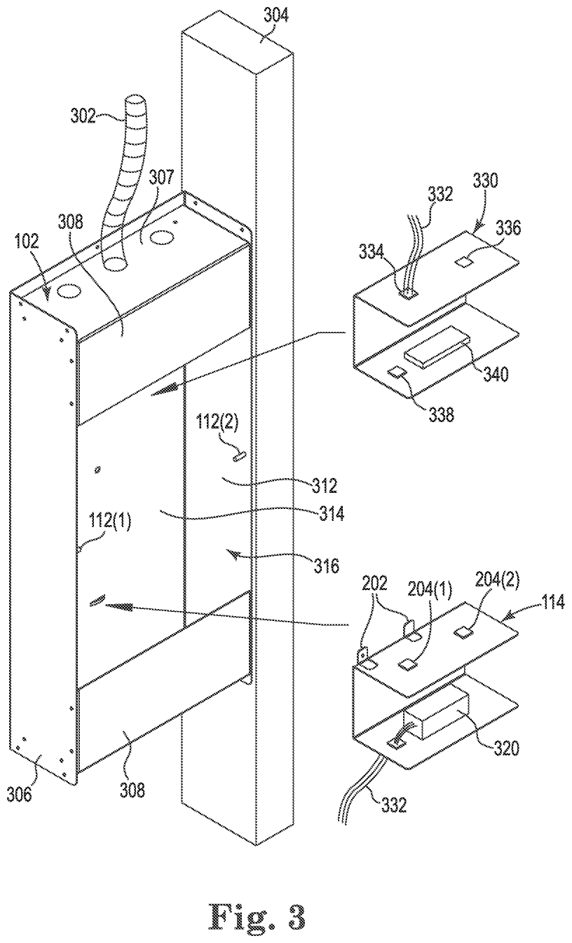

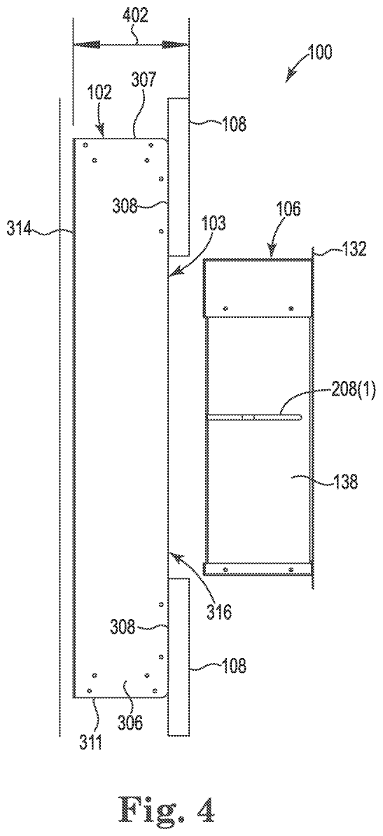

FIG. 3 is a diagram illustrating a perspective view of the back box 102 of a UV light fixture apparatus mounted to a stud 304 of a mounting surface according to an example. The back box 102 includes a left side panel 306, a top panel 307, a front panel 308, a bottom panel 311 (FIG. 4), a right side panel 312, and a back panel 314. A rectangular-shaped opening 316 is provided in the front panel 308. A power cable 302 is attached to the back box 102 at the top panel 307, and is configured to provide power from an external power source (e.g., the building mains) to elements within the back box 102. In an example, the back box 102 uses 120 VAC power. An emergency power supply may be used to provide power when power is lost to the building mains.

The back box 102 may include a cradle assembly 330, which may be attached to an interior surface of back panel 314 of the back box 102 via at least one attachment mechanism. The cradle assembly 330 includes circuitry 340, and electrical contacts 334, 336, and 338. One or more of the electrical contacts 334, 336, and 338 of the cradle assembly 330 may be electrically connected to a source of power, such as via wires 332 from power cable 302. One or more of the electrical contacts 334, 336, and 338 of the cradle assembly 330 may be connected to circuitry 340 and to electrical contacts of the fixture head apparatus 106 (FIG. 2) via wires. In an example, circuitry 340 may be voltage regulation circuitry.

In some examples, the back box 102 may also include cradle assembly 114, which may be attached to an interior surface of back panel 314 of the back box 102 via attachment mechanisms 202. The cradle assembly 114 includes circuitry 320, and electrical contacts 204(1) and 204(2). One or more of the electrical contacts 204(1) and 204(2) of the cradle assembly 114 may be electrically connected to network communications equipment (e.g., Ethernet) positioned remotely from the back box 102. One or more of the electrical contacts 204(1) and 204(2) of the cradle assembly 114 may be connected to circuitry 320 and to electrical contacts of the fixture head apparatus 106 (FIG. 2) via wires. Circuitry 320 may be connected to remote network communications equipment and/or the fixture head apparatus 106 via wires 332. In an example, circuitry 320 may be network communications circuitry, such as Ethernet communications circuitry.

FIG. 4 is a diagram illustrating a side view of a UV light fixture apparatus 100 prior to installation in a mounting surface 108 according to an example. Back box 102 is positioned behind the wall 108 and includes a front panel 308 with an opening 316 that faces the opening 103 in the wall 108. The opening 316 in the front panel 308 of the back box 102 allows the rear portion of the fixture head apparatus 106 to be received into the interior of the back box 102. In the illustrated example, the opening 103 in the wall 108 has a size that matches the size of the opening 316 in the front panel 308 of the back box 102. In an example, the opening 316 in the front panel 308 is about 12.5 inches high by about 9.5 inches wide. In other examples, other dimensions may be used.

In an example, a depth 402 of about 4.25 inches between the front surface of the wall 108 and the back panel 314 of the back box 102 is provided for flush mounting the fixture head apparatus 106 to the wall 108. After installation, the front surface of the front panel 132 is flush or nearly flush with the outer finished surface of the wall 108. After installation, the rear portion of the fixture head apparatus 106 (e.g., the portion behind the front panel 132) is positioned completely behind the outer surface of the wall 108.

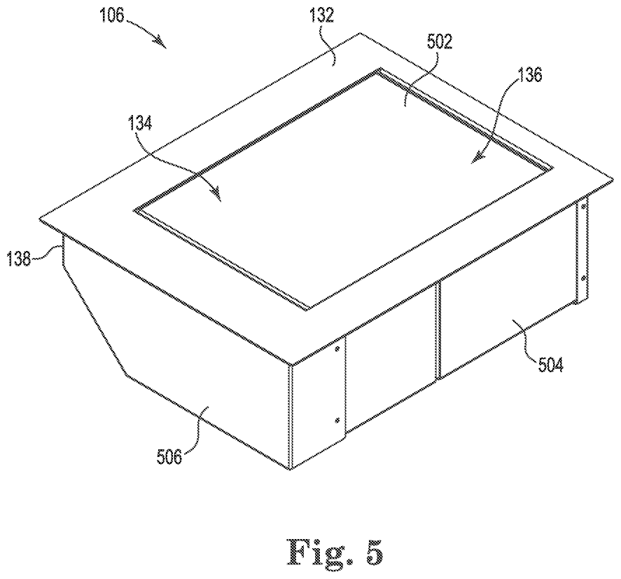

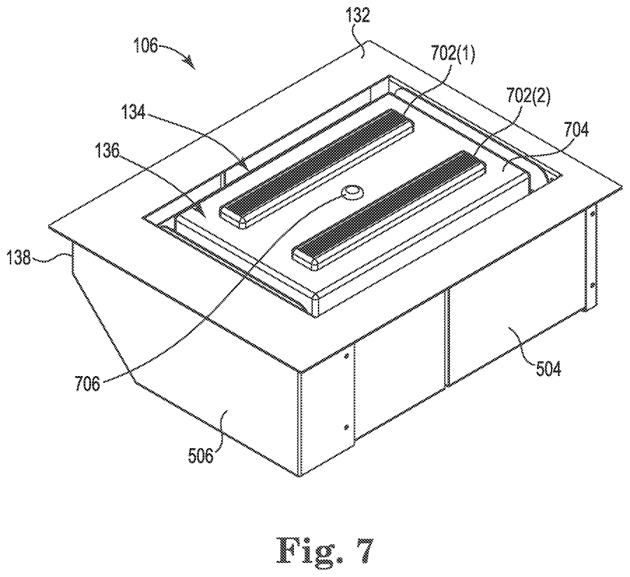

FIG. 5 is a diagram illustrating a perspective view of the fixture head apparatus 106 of a UV light fixture apparatus in a concealed position according to an example. The fixture head apparatus 106 includes front panel 132, rotatable fixture head unit 136, right side panel 504, and bottom panel 506. The rotatable fixture head unit 136 is rotatable between a concealed position (shown in FIG. 5) and an exposed position (shown in FIG. 7). In the concealed position, a cover panel 502 of the rotatable fixture head unit 136 covers the opening 134 of the front panel 132. The outer surface of the cover panel 502 is flush or nearly flush with the outer surface of the front panel 132. Matching wall finishes may be applied to the wall or other mounting surface, the front panel 132, and the cover panel 502, which conceals the fixture head apparatus 106 while it is in the concealed position and allows it to blend in to its surroundings and appear to be part of the wall.

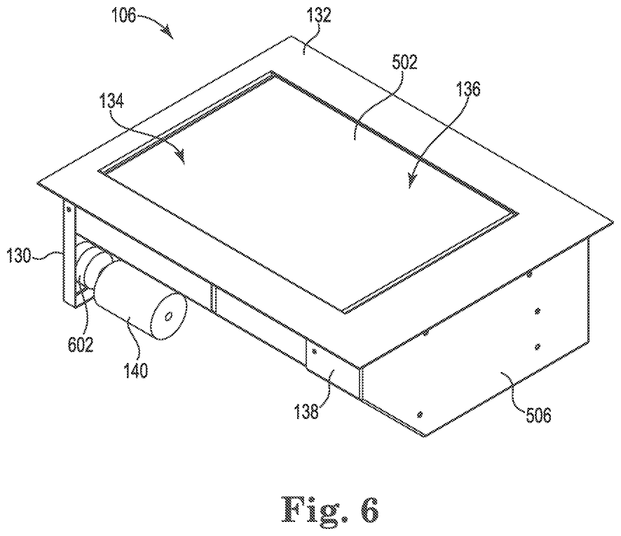

FIG. 6 is a diagram illustrating another perspective view of the fixture head apparatus 106 shown in FIG. 5 in a concealed position according to an example. Fixture head apparatus 106 includes a motor 140 coupled to a clutch 602, which is connected to the top panel 130. Motor 140 drives the rotatable fixture head unit 136 between the concealed position and the exposed position.

FIG. 7 is a diagram illustrating another perspective view of the fixture head apparatus 106 shown in FIG. 5 in an exposed position according to an example. In the exposed position, as shown in FIG. 7, a base panel 704 is positioned within the opening 134 of the front panel 132. UV light lamps 702(1) and 702(2) (collectively referred to as UV light lamps 702) are positioned parallel to each other on the base panel 704, and extend longitudinally from near one longitudinal end of the base panel 704 to near an opposite longitudinal end of the base panel 704. The UV light lamps 702 are laterally offset from one another, and a motion sensor 706 is positioned on the base panel 704 between the two UV light lamps 702 near a center of the base panel 704.

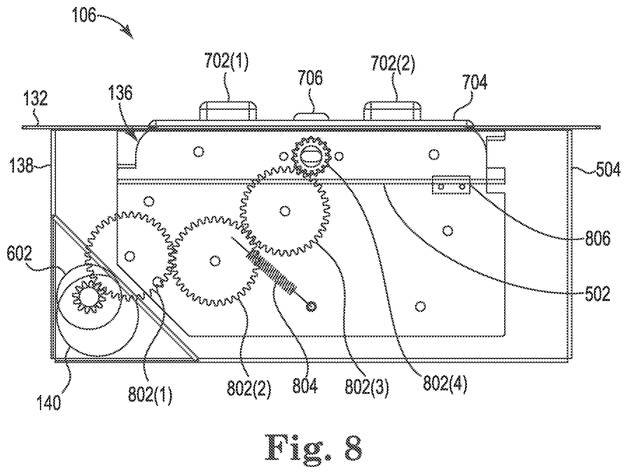

FIG. 8 is a diagram illustrating internal details of the fixture head apparatus 106 shown in FIG. 5 according to an example. As shown in FIG. 8, the fixture head apparatus 106 is in the exposed position, with the UV light lamps 702(1) and 702(2) and the motion sensor 706 exposed for operation. The fixture head apparatus 106 includes gears 802(1)-802(4) (collectively referred to as gears 802), which are coupled to and driven by motor 140. Stopping and starting of the gears is controlled by the electrically held clutch 602. Movement of the gears 802 causes rotational movement of the rotatable fixture head unit 136. The rotation occurs about the gear 802(4) in a plane that is parallel to the paper in FIG. 8.

The motor 140 may drive the gears 802 to cause the rotatable fixture head unit 136 to rotate in a clockwise direction from the concealed position in which the cover panel 502 is exposed, to the exposed position shown in FIG. 8 in which the base panel 704 is exposed. A limit switch 806 may be used to sense when the rotatable fixture head unit 136 has been completely driven into the exposed position so that the driving of the gears 802 can be stopped at that point. A spring return mechanism 804 may be used to return the rotatable fixture head unit 136 to the concealed position.

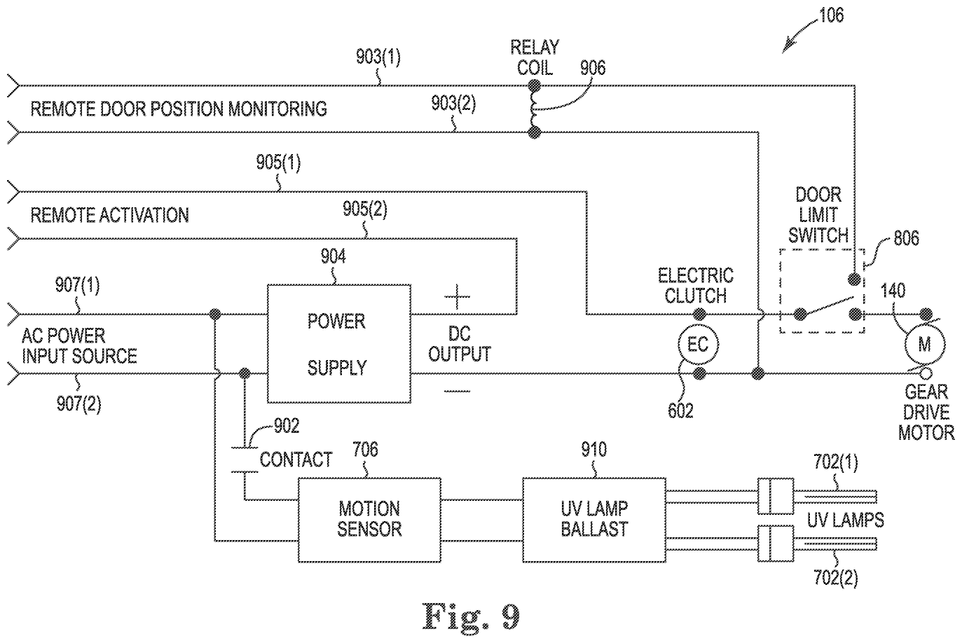

FIG. 9 is an electrical schematic diagram illustrating electrical elements of the fixture head apparatus 106 shown in FIG. 5 according to an example. The fixture head apparatus 106 includes contact 902, power supply 904, relay coil 906, electric clutch 602, door limit switch 806, gear drive motor 140, motion sensor 706, UV lamp ballast 910, and UV lamps 702(1) and 702(2). AC power input lines 907(1) and 907(2) are coupled to power supply 904 to provide AC power to the fixture head apparatus 106. Power supply 904 provides a DC output signal to drive electric clutch 602 and gear drive motor 140. Remote activation lines 905(1) and 905(2) provide signals to activate and deactivate electric clutch 602 and gear drive motor 140. Remote door position monitoring lines 903(1) and 903(2) provide signals to activate and deactivate relay coil 906, and to open and close the door limit switch 806 and thereby deactivate and activate, respectively, the gear drive motor 140. Motion sensor 706 is coupled to AC power input lines 907(1) and 907(2) and senses motion within a room containing the fixture head apparatus 106. AC power input line 907(2) is coupled to the motion sensor 706 via contact 902. UV lamp ballast 910 controls power that is supplied to the UV lamps 702(1) and 702(2). When motion sensor 706 senses motion, it sends a signal to UV lamp ballast 910 to cause the UV lamp ballast 910 to cut off the power to UV lamps 702(1) and 702(2), and thereby power them off. The motion sensor 706 may be used to over-ride the activation of the rotation of the rotatable fixture head unit 136 and the activation of the UV lamps 702(1) and 702(2).

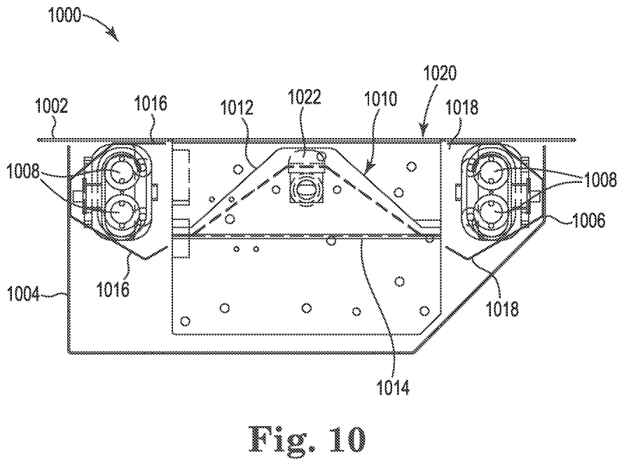

FIG. 10 is a diagram illustrating internal details of a fixture head apparatus 1000 according to another example. The fixture head apparatus 1000 includes front panel 1002, rotatable fixture head unit 1010, left side panel 1004, right side panel 1006, four UV lamps 1008, and reflectors 1016 and 1018. Two of the UV lamps 1008 are positioned adjacent the left side panel 1004 with a first reflector 1016 positioned above the two UV lamps 1008 and a second reflector 1016 positioned below the two UV lamps 1008. The two reflectors 1016 reflect light from the two UV lamps 1008 toward the rotatable fixture head unit 1010. Two other ones of the UV lamps 1008 are positioned adjacent the right side panel 1006 with a first reflector 1018 positioned above the two other UV lamps 1008 and a second reflector 1018 positioned below the two other UV lamps 1008. The two reflectors 1018 reflect light from the two other UV lamps 1008 toward the rotatable fixture head unit 1010.

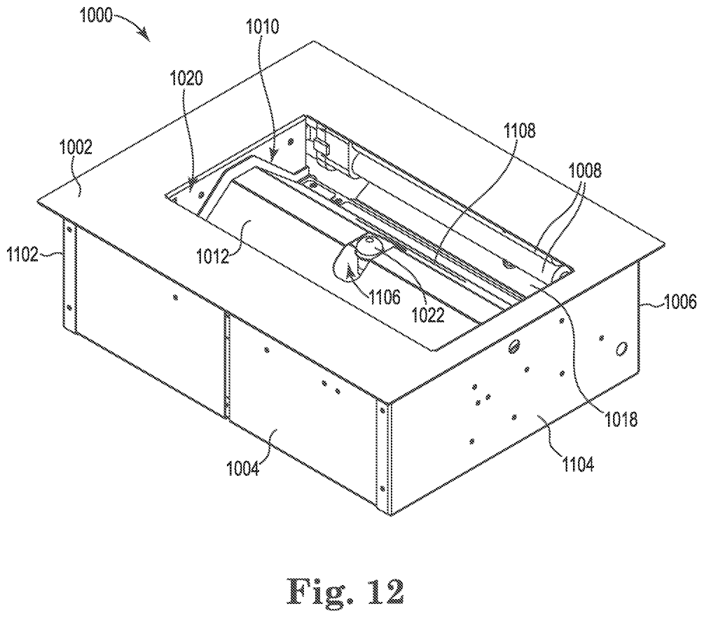

The rotatable fixture head unit 1010 is rotatable between a concealed position (which has the same appearance as shown in FIG. 5) and an exposed position (see FIG. 12). In the concealed position, a cover panel 1014 of the rotatable fixture head unit 1010 covers an opening 1020 of the front panel 1002. In the concealed position, the outer surface of the cover panel 1014 is flush or nearly flush with the outer surface of the front panel 1002. Matching wall finishes may be applied to the wall or other mounting surface, the front panel 1002, and the cover panel 1014, which conceals the fixture head apparatus 1000 while it is in the concealed position and allows it to blend in to its surroundings and appear to be part of the wall.

In the exposed position, as shown in FIG. 10, a reflector panel 1012 of the rotatable fixture head unit 1010 is positioned within the opening 1020 of the front panel 1002. The reflector panel 1012 includes a first portion that ramps upward with increasing distance from the UV lamps 1008 adjacent the left side panel 1004, and includes a second portion that ramps upward with increasing distance from the UV lamps 1008 adjacent the right side panel 1006. The reflector panel 1012 reflects light from the four UV lamps 1008 through the opening 1020 of the front panel 1002.

A stationary motion sensor 1022 is mounted between the cover panel 1014 and the reflector panel 1012 of the rotatable fixture head unit 1010, and in the illustrated example, does not move with the rotatable fixture head unit 1010. Rather, in the concealed position of the rotatable fixture head unit 1010, the motion sensor 1022 is covered by the cover panel 1014 and is not visible. In the exposed position of the rotatable fixture head unit 1010, the motion sensor 1022 is exposed and visible through the opening 1020 of the front panel 1002. In the exposed position, the UV lamps 1008 and the motion sensor 1022 are exposed for operation.

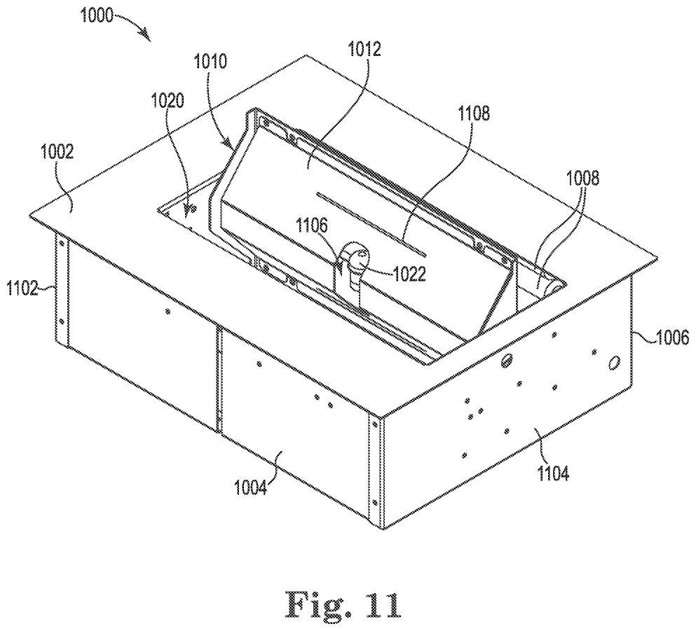

FIG. 11 is a diagram illustrating a perspective view of the fixture head apparatus 1000 shown in FIG. 10 transitioning from a concealed position to an exposed position according to an example. FIG. 12 is a diagram illustrating a perspective view of the fixture head apparatus 1000 shown in FIG. 10 in an exposed position according to an example. The fixture head apparatus includes front panel 1002, top panel 1102, left side panel 1004, right side panel 1006, bottom panel 1104, and rotatable fixture head unit 1010. As shown in FIGS. 11 and 12, the reflector panel 1012 of the rotatable fixture head unit 1010 includes an opening 1106 near a center of the reflector panel 1012. In the exposed position shown in FIG. 12, the motion sensor 1022 is positioned within the opening 1106 of the reflector panel 1012 and is visible through the opening 1020 of the front panel 1002.

At least one sanitizing slot 1108 is formed in the reflector panel 1012, which allows UV light to enter therein and sanitize internal elements of the UV light fixture apparatus 1000. In an example, a sanitizing slot 1108 is formed in each of the ramped portions of the reflector panel 1012. In an example, sanitizing slots 1108 are also formed in reflectors 1016 and 1018 (FIG. 10).

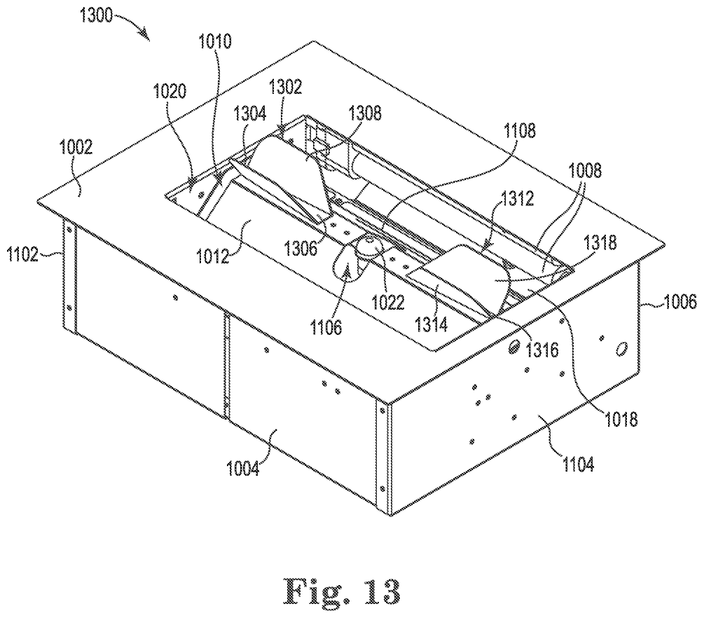

FIG. 13 is a diagram illustrating a perspective view of a fixture head apparatus 1300 in an exposed position according to another example. Fixture head apparatus 1300 according to an example is the same as fixture head apparatus 1000 (FIGS. 10-12), with the exception that compound angled mirror reflectors 1302 and 1312 have been added to the reflector panel 1012.

Reflector 1302 includes a flat base portion 1306 attached to the reflector panel 1012, and two wings 1304 and 1308 that extend upward from the opposing edges of the base portion 1306, and extend to above the front panel 1002. The wings 1304 and 1308 are each angled at about 30 degrees from vertical (i.e., vertical with respect to the base portion 1306) in opposite directions (i.e., wing 1304 is angled toward left side panel 1004, and wing 1308 is angled toward right side panel 1006 (FIG. 10)), so the wings 1304 and 1308 are angled at about 60 degrees away from each other.

Reflector 1312 includes a flat base portion 1316 attached to the reflector panel 1012, and two wings 1314 and 1318 that extend upward from the opposing edges of the base portion 1316, and extend to above the front panel 1002. The wings 1314 and 1318 are each angled at about 30 degrees from vertical (i.e., vertical with respect to the base portion 1316) in opposite directions (i.e., wing 1314 is angled toward left side panel 1004, and wing 1318 is angled toward right side panel 1006 (FIG. 10)), so the wings 1314 and 1318 are angled at about 60 degrees away from each other. Reflectors 1302 and 1312 help to reflect light from the UV lamps 1008 to provide complete room UV disinfection coverage including the floor, walls, ceiling, and air space in a room.

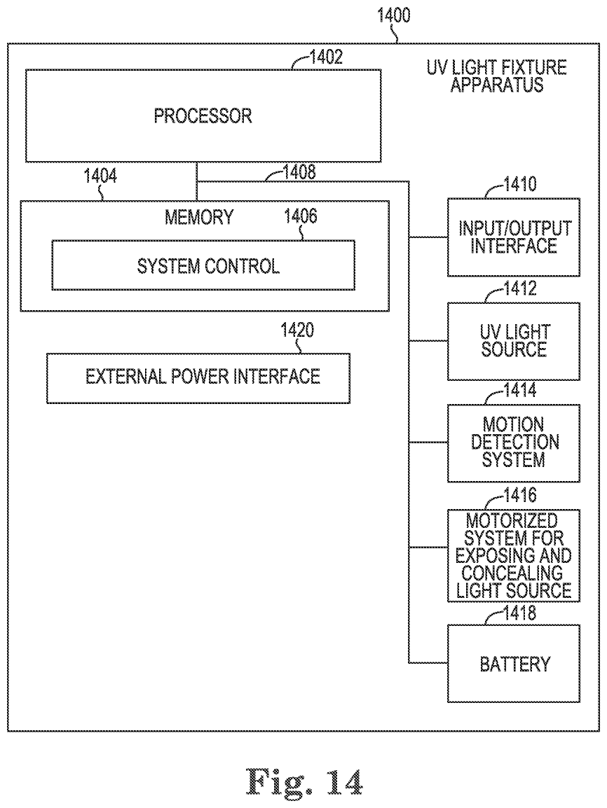

FIG. 14 is a block diagram illustrating elements of a UV light fixture apparatus 1400 according to an example. UV light fixture apparatus 1400 includes at least one processor 1402, memory 1404, input/output interface 1410, UV light source 1412, motion detection system 1414, motorized system for exposing and concealing UV light source 1416, battery 1418, and external power interface 1420. UV light fixture apparatus 1400 may also include additional or different elements than those shown in FIG. 14. In the illustrated example, processor 1402, memory 1404, input/output interface 1410, UV light source 1412, motion detection system 1414, motorized system for exposing and concealing light source 1416, and battery 1418 are communicatively coupled to each other via communication link 1408.

Processor 1402 includes a central processing unit (CPU) or another suitable processor. In one example, memory 1404 stores machine readable instructions executed by processor 1402 for operating the apparatus 1400. Memory 1404 includes any suitable combination of volatile and/or non-volatile memory, such as combinations of Random Access Memory (RAM), Read-Only Memory (ROM), flash memory, and/or other suitable memory. These are examples of non-transitory computer readable storage media. The memory 1404 is non-transitory in the sense that it does not encompass a transitory signal but instead is made up of at least one memory component to store machine executable instructions for performing techniques described herein. Memory 1404 stores system control module 1406. Processor 1402 executes instructions of module 1406 to perform techniques described herein.

In an example, the various subcomponents or elements of the apparatus 1400 may be embodied in a plurality of different systems, where different modules may be grouped or distributed across the plurality of different systems. To achieve its desired functionality, apparatus 1400 may include various hardware components. Among these hardware components may be a number of processing devices, a number of data storage devices, a number of peripheral device adapters, and a number of network adapters. These hardware components may be interconnected through the use of a number of busses and/or network connections. The processing devices may include a hardware architecture to retrieve executable code from the data storage devices and execute the executable code. The executable code may, when executed by the processing devices, cause the processing devices to implement at least some of the functionality disclosed herein.

An example of the present disclosure is directed to an ultraviolet (UV) light fixture apparatus, which includes a fixture head apparatus including a front panel, a rotatable fixture head unit, and at least one UV lamp, wherein the front panel is to be mounted substantially flush with a mounting surface with the rotatable fixture head unit positioned behind the front panel within a recess behind the mounting surface. The UV light fixture apparatus includes a motor to cause rotation of the rotatable fixture head unit to switch the rotatable fixture head unit between a concealed state in which the at least one UV lamp is not visible and an exposed state in which the at least one UV lamp is visible through an opening in the front panel.

The rotatable fixture head unit may include a base panel, and the at least one UV lamp may include at least two UV lamps mounted on the base panel. The rotatable fixture head unit may include a motion sensor mounted on the base panel.

The rotatable fixture head unit may include a reflector panel to reflect light from the at least one UV lamp through the opening in the front panel. The reflector panel may include at least one sanitizing slot that allows UV light to enter and sanitize internal elements of the UV light fixture apparatus. The UV light fixture apparatus may further include a stationary motion sensor that is covered by the rotatable fixture head unit in the concealed state and is exposed in the exposed state. The reflector panel may include reflectors to reflect light from the at least one UV lamp to provide complete room UV disinfection coverage.

The UV light fixture apparatus may include a motion sensor, and a controller to control the motor to control the rotation of the rotatable fixture head unit and to control operation of the at least one UV lamp based on signals output by the motion sensor. The controller may turn off the at least one UV lamp in response to the motion sensor sensing motion. The controller may cause the rotatable fixture head unit to switch from the exposed state to the concealed state in response to the motion sensor sensing motion.

In the concealed state, a cover panel of the rotatable fixture head unit may cover the opening in the front panel and may be substantially flush with the front panel and the mounting surface. The cover panel and the front panel may have a finish that matches a finish of the mounting surface.

The UV light fixture apparatus may further include a back box to be installed completely within the recess behind the mounting surface, wherein the back box is to be connected to an AC power source to provide power to the fixture head apparatus, and wherein the back box includes an opening to receive a back portion of the fixture head apparatus into an interior of the back box. The UV light fixture apparatus may further include a plurality of gears, wherein the motor is to drive the gears to cause rotation of the rotatable fixture head unit. The UV light fixture apparatus may further include an electric clutch, wherein the motor is coupled to the gears via the electric clutch. The UV light fixture apparatus may be positioned in a room, and the at least one UV lamp may emit UV light to disinfect the room.

Another example of the present disclosure is directed to an ultraviolet (UV) light fixture apparatus, which includes a fixture head apparatus including a front panel, a movable fixture head unit, and at least one UV lamp, wherein the front panel is to be mounted substantially flush with a mounting surface in a room with the movable fixture head unit positioned behind the front panel within a recess behind the mounting surface. The UV light fixture apparatus includes a motorized system to cause movement of the movable fixture head unit to switch the movable fixture head unit between a concealed state in which the at least one UV lamp is not visible and an exposed state in which the at least one UV lamp is visible through an opening in the front panel and is to emit UV light to disinfect the room. The UV light fixture apparatus includes a motion sensor to generate a signal to turn off the at least one UV lamp when the motion sensor senses motion within the room.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

What is claimed is:

1. An ultraviolet (UV) light fixture apparatus, comprising:
a fixture head apparatus including a front panel, a rotatable fixture head unit, and at least one UV lamp, wherein the front panel is to be mounted substantially flush with a mounting surface with the rotatable fixture head unit positioned behind the front panel within a recess behind the mounting surface; and
a motor to cause rotation of the rotatable fixture head unit to switch the rotatable fixture head unit between a concealed state in which the at least one UV lamp is not visible and an exposed state in which the at least one UV lamp is visible through an opening in the front panel.

2. The UV light fixture apparatus of claim 1, wherein the rotatable fixture head unit includes a base panel, and wherein the at least one UV lamp includes at least two UV lamps mounted on the base panel.

3. The UV light fixture apparatus of claim 2, wherein the rotatable fixture head unit includes a motion sensor mounted on the base panel.

4. The UV light fixture apparatus of claim 1, wherein the rotatable fixture head unit includes a reflector panel to reflect light from the at least one UV lamp through the opening in the front panel.

5. The UV light fixture apparatus of claim 4, wherein the reflector panel includes at least one sanitizing slot that allows UV light to enter and sanitize internal elements of the UV light fixture apparatus.

6. The UV light fixture apparatus of claim 4, and further comprising a stationary motion sensor that is covered by the rotatable fixture head unit in the concealed state and is exposed in the exposed state.

7. The UV light fixture apparatus of claim 4, wherein the reflector panel includes reflectors to reflect light from the at least one UV lamp to provide complete room UV disinfection coverage.

8. The UV light fixture apparatus of claim 1, and further comprising:
a motion sensor; and
a controller to control the motor to control the rotation of the rotatable fixture head unit and to control operation of the at least one UV lamp based on signals output by the motion sensor.

9. The UV light fixture apparatus of claim 8, wherein the controller is to turn off the at least one UV lamp in response to the motion sensor sensing motion.

10. The UV light fixture apparatus of claim 9, wherein the controller is to cause the rotatable fixture head unit to switch from the exposed state to the concealed state in response to the motion sensor sensing motion.

11. The UV light fixture apparatus of claim 1, wherein, in the concealed state, a cover panel of the rotatable fixture head unit covers the opening in the front panel and is substantially flush with the front panel and the mounting surface.

12. The UV light fixture apparatus of claim 11, wherein the cover panel and the front panel have a finish that matches a finish of the mounting surface.

13. The UV light fixture apparatus of claim 1, and further comprising:
a back box to be installed completely within the recess behind the mounting surface, wherein the back box is to be connected to an AC power source to provide power to the fixture head apparatus, and wherein the back box includes an opening to receive a back portion of the fixture head apparatus into an interior of the back box.

14. The UV light fixture apparatus of claim 1, and further comprising:
a plurality of gears, wherein the motor is to drive the gears to cause rotation of the rotatable fixture head unit.

15. The UV light fixture apparatus of claim 14, and further comprising:
an electric clutch, wherein the motor is coupled to the gears via the electric clutch.

16. The UV light fixture apparatus of claim 1, wherein the UV light fixture apparatus is to be positioned in a room, and wherein the at least one UV lamp is to emit UV light to disinfect the room.

17. An ultraviolet (UV) light fixture apparatus, comprising:
a fixture head apparatus including a front panel, a movable fixture head unit, and at least one UV lamp, wherein the front panel is to be mounted substantially flush with a mounting surface in a room with the movable fixture head unit positioned behind the front panel within a recess behind the mounting surface;
a motorized system to cause movement of the movable fixture head unit to switch the movable fixture head unit between a concealed state in which the at least one UV lamp is not visible and an exposed state in which the at least one UV lamp is visible through an opening in the front panel and is to emit UV light to disinfect the room; and
a motion sensor to generate a signal to turn off the at least one UV lamp when the motion sensor senses motion within the room.
